(19) United States
(12) Patent Application Publication
(10) Pub. No.: US 2009/0093655 A1
(43) Pub. Date: Apr. 9, 2009

(54) METHOD AND DEVICE FOR PRODUCING AROMATIC AMINES BY HETEROGENEOUS CATALYZED HYDRATION

(75) Inventors: Stephan Schubert, Leverkusen (DE); Ralph Schellen, Dormagen (DE); Leslaw Mleczko, Dormagen (DE); Stephan Laue, Koln (DE); Peter Lehner, Mulheim/Ruhr (DE)

Correspondence Address: NORRIS, MCLAUGHLIN & MARCUS, P.A., 875 THIRD AVE, 18TH FLOOR, NEW YORK, NY, 10022, US

(73) Assignee: BAYER TECHNOLOGY SERVICES GMBH, Leverkusen (DE)

(21) Appl. No.: 12/282,363

(22) PCT Filed: Mar. 1, 2007
(86) PCT No.: PCT/EP2007/001751

§ 371 Date: Sep. 30, 2008

(30) Foreign Application Priority Data
Mar. 14, 2006 (DE) 10 2006 011 497.3

(51) Int. Cl. C07C 209/32; B01J 19/24

(52) U.S. Cl. 564/411; 422/198

(57) ABSTRACT

The present invention relates to a process and an apparatus for preparing aromatic amines by means of a heterogeneously catalysed hydrogenation, wherein the catalyst required for the reaction is applied to the interior wall of one or more reaction channels which are cooled from the outside.

The invention relates to a process for preparing aromatic amines by catalytic hydrogenation of aromatic nitro compounds in the presence of a catalyst applied to the interior wall of a reaction channel which is cooled from the outside.

Aromatic amines are important intermediates which have to be prepared inexpensively in large quantities. High space-time yields and long catalyst operating lives are therefore critical to the economics of the process. The hydrogenation of nitroaromatics is a strong exothermic reaction. The removal and utilisation of the energy content of the heat of reaction is therefore an important aspect of the preparation of aromatic amines.

Various reactors are suitable for the gas-phase hydrogenation of nitroaromatics. Thus, for example, U.S. Pat. No. 3,136,818 describes a process in which the reaction is carried out in a fluidized bed. The effective removal of heat in this mode of operation is at the expense of problems caused by the nonuniform residence time distribution (breakthrough of the nitroaromatics) and by catalyst attrition.

Other processes utilise catalysts fixed in place in fixed beds. This arrangement enables the reaction to be carried out with a very narrow residence time distribution and enables the problems of catalyst attrition to be circumvented. However, a known technical problem here is the formation of hot spots, i.e. regions of local overheating in the fixed-bed catalyst. These lead to increased deactivation of the catalyst by carbonization and can in an extreme case permanently damage the catalyst.

In adiabatic operation of the fixed-bed reactor, the problem of heat removal is circumvented by the heat liberated being completely taken up by the gas stream. Such a process, which is associated with a simple construction and an ability of the individual apparatuses to be scaled up easily, is described, for example, in EP 0 696 574. However, to keep the adiabatic temperature increase within limits, very large gas streams have to be circulated in the adiabatic mode of operation.

DE2849002 describes a process for the reduction of nitro compounds in the presence of fixed palladium-containing multicomponent support catalysts in cooled shell-and-tube reactors. The catalyst consists essentially of from 1 to 20 g of palladium, from 1 to 20 g of vanadium and from 1 to 20 g of lead per litre of α-Al₂O₃. It has been found to be advantageous for the active components to be present in precipitated form as close as possible to the surface of the catalyst in a very sharply defined zone and for no active components to be present in the interior of the support material. A disadvantage of the gas-phase hydrogenation described in DE 2 849 002 is the low specific weight hourly space velocity over the catalysts, which is attributable essentially to unsatisfactory heat removal. The indicated weight hourly space velocities are from about 0.4 to 0.5 kg/(l-h). The weight hourly space velocity is defined here as the amount of nitroaromatic in kg which is passed through per litre of catalyst bed in one hour. The low weight hourly space velocity over the catalyst is associated with an unsatisfactory space-time yield in industrial processes for preparing aromatic amines. Furthermore, the selectivities at the beginning of a period of operation are significantly lower than towards the end, which leads to decreases in yield and problems in the work-up of the crude product.

The weight hourly space velocity over the catalyst in isothermally operated reactors can only be increased when the heat liberated in the reaction can be removed efficiently. WO 98/25881 describes the use of inert materials for diluting the catalyst bed in the preparation of aromatic amines. The dilution broadens the reaction zone and thus increases the area available for heat transfer. This procedure enables the hot spot temperature to be decreased or the possible weight hourly space velocity of the nitroaromatic to be increased at a constant hot spot temperature. However, the dilution results in a decrease in the operating life of the bed. In the example described in WO 98/25881, the productivity of the diluted bed was significantly less than the productivity of the undiluted bed because of the short operating lives despite a higher weight hourly space velocity.

In Chemical Engineering Science 56 (2001) pp. 1347-1353, Klemm et al. describe the production of a laboratory catalytic wall reactor for determining the deactivation kinetics of the catalytic hydrogenation of nitrobenzene to aniline. The direct application of the 100 μm-400 μm thick catalyst layer to the interior wall of a metal tube which has an internal diameter of 10 mm and is cooled from the outside enables isothermic reaction conditions to be achieved. However, scale-up of the wall reactor described which has tubes which have to be individually coated and subsequently assembled to form an industrial reactor is not practicable. A further disadvantage of the wall reactor described is the low proportion of solids based on the tube volume of 4%-15%, which in industrial implementation of the concept would lead to very large reactor dimensions.

DE 1 0347 439 describes a process for preparing aromatic amines, in which a catalyst comprising a monolithic support and a thin catalytically active coating is used. An advantage here is that a higher selectivity than in comparable fixed beds can be achieved as a result of the thin catalytically active layer. However, a disadvantage is that the monolithic supported catalyst described cannot be cooled from the outside but only adiabatically, i.e. generally with a large circulating gas stream, because of its poor radial heat conduction, which leads to a complicated mode of operation of the process and further costs.

It is therefore an object of the present invention to provide a process for preparing aromatic amines by catalytic hydrogenation of aromatic nitro compounds, which reduces the formation of hot spots and allows a high space-time yield, a longer operating life and higher selectivity.

The invention provides a process for preparing aromatic amines by catalytic hydrogenation of aromatic nitro compounds, which is characterized in that the catalyst required for the reaction is applied to the interior wall of one or more reaction channels which are cooled from the outside. It has surprisingly been found that the formation of hot spots during the process of the invention can is effectively reduced and a higher space-time yield combined with higher selectivity is made possible as a result.

The cooling medium which is in thermal contact with the reaction channel is preferably likewise conveyed through at least two cooling medium channels which are essentially parallel to one another and through which flow occurs in cocurrent, in countercurrent or in cross-current relative to the main flow direction in the reaction channel.

Cooling media used are salt melts, steam, organic compounds or metal melts, preferably salt melts, steam or heat transfer fluids, particularly preferably a mixture of potassium nitrate, sodium nitrite and sodium nitrate, dibenzyltoluene or a mixture of diphenyl oxide and biphenyl.

In a preferred embodiment of the process, the cooling medium or media are conveyed in cross-current relative to the main flow direction in the reaction channel. The cooling medium is usually divided between at least two essentially parallel cooling medium channels and the cooling medium channels can have different materials properties, flow velocities, throughputs or temperatures.

The catalyst is applied to the interior wall of the reactor channel in a layer having a thickness of from 5 to 1000 μm, preferably from 10 to 500 μm, particularly preferably from 20 μm to 200 μm. The application can be carried out according to essentially any known technology. Preference is given to using methods in which a plurality of reaction channels are coated with catalysts by means of a single coating step. Particular preference is given to using methods in which the catalyst is applied as washcoat to the interior wall of the reaction channels. The catalyst is usually applied simultaneously to the interior wall of at least two reaction channels which are cooled from the outside.

The catalytically active coating for the hydrogenation of aromatic nitro compounds in the gas phase preferably contains metals of groups VIIIa, Ib, IIb, IVa, Va, VIa, IVb and Vb of the Periodic Table of the Elements (Mendeleev, Zeitschrift für Chemie 12, 405-6, 1869) as catalytically active components. Preferred metals are Pd, Pt, Cu and Ni. The catalytically active component can be applied to a support. Suitable support substances are ceramic materials such as Al₂O₃, SiO₂, TiO₂ or zeolites, and also graphite or carbon. The support substance is preferably finely milled. To achieve uniform coating, the volume-based particle size d₉₀ of the preferably milled support substance should preferably be less than 50 μm, particularly preferably less than 10 μm. Particular preference is given to using the catalyst described in DE 2 849 002 as catalytically active coating.

Preference is given to operating at least two reaction channels under identical reaction conditions.

The following inventive reactor, which is likewise subject matter of the present patent application, is suitable for the process, without the process being restricted thereto.

The reactor for the catalytic hydrogenation of aromatic nitro compounds by the process of the invention comprises one or more reaction channels to whose interior walls the catalyst required for the reaction has been applied and which are cooled from the outside.

The proportion of the total volume of the apparatus which is made up by the catalyst is usually from 1% to 50%, preferably from 5% to 35%, particularly preferably from 10% to 25%.

The reaction channels have a round or rectangular cross section having a hydraulic diameter, defined as the ratio of four times the internal cross-sectional area to the internal diameter, from 0.05 mm to 100 mm, preferably from 0.1 mm to 10 mm, particularly preferably from 0.5 mm to 2 mm, and a length of from 0.02 m to 5.0 m, preferably from 0.1 m to 1.0 m, particularly preferably from 0.2 m to 0.7 m.

At least two, preferably from 20 000 to 200 000 000, reaction channels, preferably of the same geometry, are preferably arranged in parallel. In a particular embodiment, the reaction channels are arranged in one or more plates for reaction channels, so that this plate is in thermal contact with at least one set of parallel cooling medium channels, preferably also in one or more plates. Preference is here given to at least two, particularly preferably from 200 to 20 000, reaction channels per plate being arranged in parallel (FIG. 1). Furthermore, preference is given to at least two, particularly preferably from 100 to 10 000, plates comprising reaction channels being arranged alternately with a comparable number of plates comprising cooling medium channels in parallel above one another (FIGS. 2 and 3).

In a particular embodiment of the reactor, the superposed alternating plates comprising reaction channels and the cooling medium channels are subdivided into individual interchangeable modules (FIGS. 4 and 5). In a particular embodiment of the process, at least two modules of superposed planes of reaction channels and cooling medium channels are operated in parallel under identical reaction conditions, so that an individual module can, owing to its construction, be removed from the process, added to the process or replaced without interrupting the operation of the other modules.

The temperature in the reaction channels is kept at a very constant temperature by means of the cooling medium channels through which cooling medium flows. When a temperature control zone is used, this temperature is in the range from 200° C. to 500° C., preferably from 220° C. to 400° C., particularly preferably from 240° C. to 330° C.

The temperature in the reaction channel can be monitored during the process by means of sensors and the flow rate or temperature of the cooling medium in the cooling medium channels can be adjusted if required. The sensors can be arranged either in the region of the cooling medium or in the region of the reaction channel, preferably in the inlet and outlet for the cooling medium.

The process of the invention is preferably carried out at pressures of from 1 to 30 bar, particularly preferably from 1 to 20 bar, very particularly preferably from 1 to 15 bar. The temperature of the feed gas mixture upstream of the reactor inlet is preferably from 200 to 400° C. Hydrogen and aromatic nitro compound are fed to the reactor in a molar ratio of hydrogen to nitro group of preferably from 3:1 to 100:1. As aromatic nitro compound, it is possible to hydrogenate, in particular, compounds of the following formula:

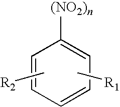

where R₁ and R₂ are identical or different and are each hydrogen or C₁- to C₄-alkyl, in particular methyl or ethyl, and n=1 or 2. Preference is given to hydrogenating nitrobenzene or the isomeric nitrotoluenes by the process of the invention.

The process can be carried out continuously or batchwise, preferably continuously.

The process of the invention can be carried out on an industrial scale. The space-time yield (kg of aniline per kg of catalyst and h) is in the range from 0.1 to 100 kg/kg/h, preferably from 1 to 50 kg/kg/h and particularly preferably from 2 to 25 kg/kg/h.

The reaction channel coated with catalysts which is used according to the invention for preparing aromatic amines displays significant advantages over conventional catalyst beds known from the prior art.

Firstly, the pressure drop in the coated reaction channel is significantly lower than that in catalyst beds at a comparable flow velocity. Conversely, the reaction mixture can flow through the coated reaction channel at a far higher velocity at the same pressure drop.

Secondly, owing to the small thickness of the catalyst layer applied to the interior wall and the good heat transfer between the applied catalyst and the channel wall which is cooled by means of an external cooling medium, the heat removal achieved is so high that the heat of reaction liberated in the reaction can be removed virtually completely from the reaction channel, so that hot spots can be avoided and longer operating lives can be achieved.

The very thin catalytically active coating also offers a further advantage. If the catalytically active components are deposited in a very thin layer, the influence of diffusion is far less than in the case of all-active catalysts. If the main reaction is accompanied by subsequent reactions, a higher selectivity can be achieved using these very thin catalytically active coatings. In addition, application in a thin layer can bring advantages in terms of the selectivity to the desired product.

Compared to the described prior art, the use of a modular reactor concept (FIG. 4) in which the cooled reactor comprises a plurality of modules operated in parallel is advantageous when immobilized catalysts are used. This construction makes it possible to replace individual modules when catalyst replacement is necessary and thus to reduce the production downtime significantly. The production downtime can possibly also be partly or completely avoided by running the other reactor modules at a higher weight hourly space velocity for a short time. Owing to the ease of handling, the modular construction of a microreactor represents a particularly useful and therefore preferred variant.

### FIGURES

Working examples of the subject matter of the invention are shown in FIG. 1 to 5 without the invention being restricted thereto.

FIG. 1: Structured plate with parallel reaction channels (1).

FIG. 2: Module comprising alternately superposed, structured plates for reaction channels (1) and cooling medium channels (2) in cross-current.

FIG. 3: Cross section of a module comprising alternately superposed, structured plates for reaction channels (1) and cooling medium channels (2) in cocurrent or countercurrent.

FIG. 4: Arrangement of interchangeable modules operated in parallel.

FIG. 5: Connections of the reaction channels and the cooling medium channels of two modules operated in parallel which can be replaced without interrupting the operation of the other modules.

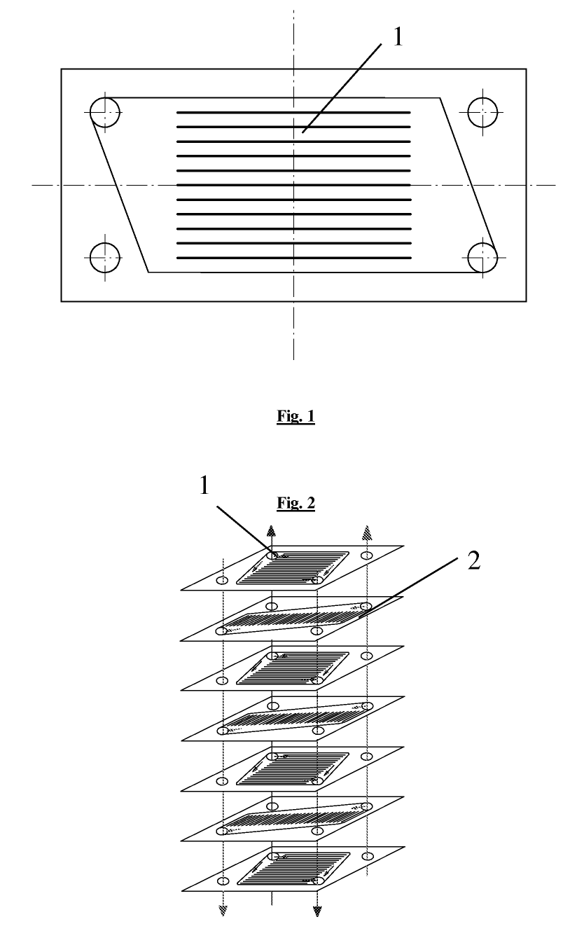

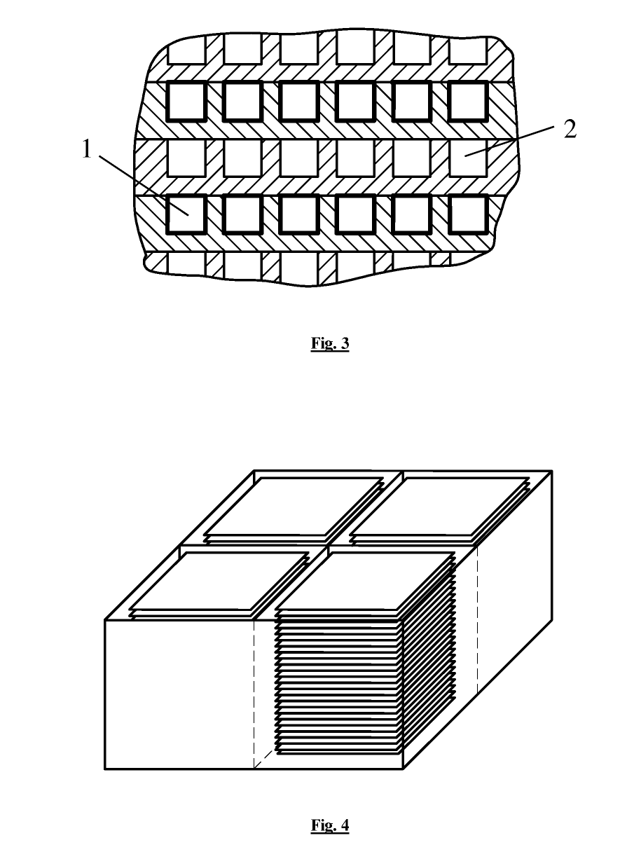

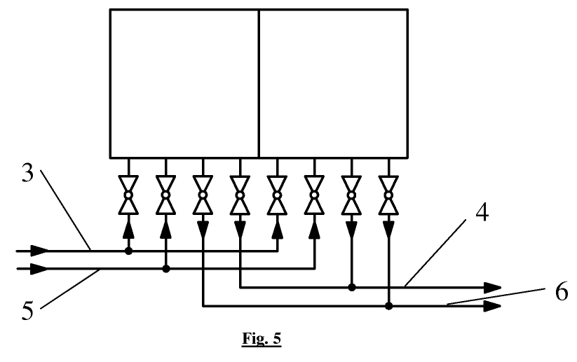

### REFERENCE NUMERALS

1—Reaction channels
2—Cooling medium channels
3—Feed to the reaction channels
4—Discharge from the reaction channels
5—Feed to the cooling medium channels
6—Discharge from the cooling medium channels

### EXAMPLES

The following examples illustrate the present invention without the invention being restricted thereto.

### Example 1 (Comparative Example)

18 g of a catalyst produced as described in Example 1 of DE-A 28 49 002 A1, which had been diluted with 43.7 g of SiC, were introduced into a reactor tube which had an internal diameter of about 26 mm and was thermostated by means of oil (240° C.). The bed height was 300 mm. The temperature of the inflowing reaction gas, consisting of 180 g/h of nitrobenzene, 200 l/h of hydrogen and 100 l/h of nitrogen, was about 240° C., and the pressure corresponded to atmospheric pressure. The nitrobenzene conversion decreased continuously with time. The mean conversion at the weight hourly space velocity set, viz. 10 g of nitrobenzene per g of catalyst per hour, averaged over the first two hours was 91.6%, corresponding to a space-time yield of 13.8 g of aniline per kg of catalyst per hour. The mean selectivity over the period of time was 99.4%.

### Example 2

### Hydrogenation in a Cooled Catalyst-Coated Reaction Channel

For the hydrogenation of nitrobenzene, two reactor modules which each comprised 28 parallel reaction channels and were thermostated by means of oil (240° C.) were connected in series. The reaction channels each had a length of 50 mm, a width of 0.5 mm and a height of 0.8 mm and were coated on the inside with a catalyst suitable for the hydrogenation of nitrobenzene. A catalyst which had been produced as described in Example 1 in DE-A 28 49 002 and had been milled to a particle size fraction with d₉₀<10 μm served as basis. The total mass of immobilized catalyst in the first reactor module was 78.2 mg, and the total mass of that in the second reactor module was 76.6 mg. The temperature of the inflowing reaction gas, consisting of 3 g/h of nitrobenzene and 6 ml/h of hydrogen, was about 240° C. and the pressure corresponded to atmospheric pressure. The nitrobenzene conversion decreased continuously with time. The mean conversion at the weight hourly space velocity set, viz. 19.4 g of nitrobenzene per g of catalyst per hour, averaged over the first two hours was 97.2%, corresponding to a space-time yield of 28.4 g of aniline per kg of catalyst per hour. The mean selectivity over the period of time was 99.7%. The temperatures between the two reactor modules and also downstream of the second reactor module were measured continuously, with no temperature increase being found.

Gas-chromatographic analyses of the products (means over the first 2 hours of operation)

| Component | | Example 1 | Example 2 |
|---|---|---|---|
| Benzene | [%] | 0.157 | 0.024 |
| Cyclohexylamine | [%] | 0.003 | 0.002 |
| Cyclohexanol | [%] | 0.002 | 0.090 |
| Cyclohexanone | [%] | 0.004 | 0.058 |
| Aniline | [%] | 91.012 | 96.901 |
| Phenol | [%] | 0.022 | 0.009 |
| Nitrobenzene | [%] | 8.409 | 2.774 |
| N,N-Diethyl-m-toluedene | [%] | 0.000 | 0.002 |
| o-Phenyldiamine | [%] | 0.001 | 0.000 |
| m-Phenyldiamine | [%] | 0.007 | 0.000 |
| o-Aminophenol | [%] | 0.000 | 0.058 |
| m-Phenyldiamine | [%] | 0.000 | 0.005 |
| m-Phenylcyclohexylimine | [%] | 0.001 | 0.000 |
| N-Cyclohexylaniline | [%] | 0.039 | 0.011 |
| o-Aminobiphenyl | [%] | 0.000 | 0.027 |
| Diphenylamine | [%] | 0.338 | 0.004 |

1. Process for preparing aromatic amines by catalytic hydrogenation of aromatic nitro compounds, wherein said hydrogenation is carried out in one or more reaction channels and the catalyst required for the reaction is applied to the interior wall of one or more reaction channels and said reaction channels are cooled from the outside.

2. Process according to claim 1, wherein the cooling medium which is in thermal contact with the reaction channel is conveyed in cocurrent, in countercurrent or cross-current relative to the main flow direction in the reaction channel.

3. Process according to claim 1, wherein the cooling medium is divided over at least two essentially parallel flow channels.

4. Process according to claim 1, wherein one or more cooling media are conveyed in cross-current relative to the flow direction in the reaction channel, divided between at least two essentially parallel cooling medium channels and the cooling medium channels have different materials properties, flow velocities, throughputs or temperatures.

5. Process according to claim 1, wherein the catalyst applied to the interior wall of the reaction channel has a layer thickness of from 5 μm to 1000 μm.

6. Process according to claim 1, wherein the catalyst is applied simultaneously to the interior wall of at least two reaction channels which are cooled from the outside.

7. Apparatus for carrying out chemical reactions, comprising one or more reaction channels to whose interior walls a catalyst required for the reaction is applied and which are cooled from the outside.

8. Apparatus according to claim 7, wherein the reaction channel has a round or rectangular cross section and a hydraulic diameter of from 0.05 mm to 100 mm.

9. Apparatus according to claim 7, wherein at least two reaction channels of identical geometry are arranged in parallel.

10. Apparatus according to claim 7, wherein the reaction channels are arranged in one plane and in that this plane is in thermal contact with at least one parallel space through which cooling medium flows.

11. Apparatus according to claim 7, comprising at least two planes of reaction channels arranged alternately with spaces through which cooling medium flows in parallel above one another.

12. Apparatus according to claim 7, wherein the proportion of the total volume of the apparatus which is made up by catalyst is from 1% to 50%.
